(19) United States
(12) Patent Application Publication
(10) Pub. No.: US 2020/0375590 A1
(43) Pub. Date: Dec. 3, 2020

(54) MEDICAL DEVICE WITH GRASPING MECHANISM

(71) Applicant: SafePath Medical, Inc., Amesbury, MA (US)

(72) Inventors: Joseph P Lane, Amesbury, MA (US); Michael J. Boss, Baltimore, MD (US); David P. Dolan, Londonderry, NH (US); Brandon J. Rodriguez, Lawrence, MA (US)

(21) Appl. No.: 16/959,754

(22) PCT Filed: Jan. 8, 2019
(86) PCT No.: PCT/US2019/012732

§ 371 Date: Jul. 2, 2020

Related U.S. Application Data
(60) Provisional application No. 62/614,946, filed on Jan. 8, 2018.

(51) Int. Cl. A61B 17/04; A61B 17/062

(52) U.S. Cl. CPC A61B 17/0469; A61B 2017/0608; A61B 17/062; A61B 17/0467

(57) ABSTRACT

A device for suturing tissue includes a handle and a suturing mechanism coupled to the handle and being configured to suture the tissue with a suture element. The device further includes a suture grasping mechanism coupled to the housing and being configured to move between an open position for receiving the suture element and a closed position for grasping and holding the suture element to permit manipulation of the suture elements, such as tying a knot.

### CROSS-REFERENCE TO RELATED APPLICATION

This application is based on and claims priority to U.S. Provisional Patent Application 62/614,946, filed Jan. 8, 2018, the entire contents of which is incorporated by reference herein as if expressly set forth in its respective entirety herein.

### BACKGROUND

Needles and suture are used throughout the healthcare industry for indications such as wound and incision closure, securing catheters, and affixing implantable meshes, annuloplasty rings, and other medical apparatus. These sutures are used on the surface of the patient's skin as well as through laparoscopic, endoscopic, and surgical procedures. Handheld needle drivers are often used to facilitate suture knot tying in a variety of these suture applications. Because suture tying must be fast and easy, there is a need to make suturing devices with intuitive knot tying features. A medical device that can be used to easily suture tissue and knot the suture will be valuable to physicians, surgeons, nurses, physician assistants, military personnel, and other clinical and non-clinical users of suture.

### SUMMARY

In one embodiment, a device for suturing tissue according to the present invention includes a handle including a housing having a distal end and an opposite proximal end. The device also includes at least one actuator for affecting the needle and suture or suture alone, and a suture grasping mechanism (device) to assist the user in knotting suture. This grasping mechanism can be coupled or integral to the housing and is designed to grasp, release, tie or affect the suture in some beneficial manner It may be located at the proximal end of the device or at some other functional location for the user. The suturing device may utilize a pre-loaded needle and suture or a user-loaded needle and suture. It may also be a disposable device or a system utilizing a reusable handle and disposable needles or needle cartridges. The device may also feature a cutter for trimming the suture.

In a second embodiment, a device for suturing tissue includes a handle including a housing having a distal end and an opposite proximal end and a suturing needle for advancing a suture through the tissue. The suturing needle has a first pointed end and an opposite second end. The device includes at least one actuator for affecting the needle and a suture grasping mechanism for grasping, releasing, tying and affecting the suture. The device may also feature a cutter for trimming the suture.

In a third embodiment, a medical device that is not a needle-based suturing device but is used in a procedure that involves suturing tissue, can be configured with a suture grasping mechanism to grasp, release, tie, or affect the suture, and also a cutter mechanism useful for cutting suture that has been utilized in a medical procedure.

The suture grasping mechanism in the three embodiments described above comprise an elongate stationary jaw and an elongate movable jaw, which when actuated by the user, contacts the stationary jaw and grips the suture between the jaw faces. The movable jaw will separate from the stationary jaw when the actuation force is removed or reversed, thereby releasing the suture. The elongate nature of the jaws permits the user to create loops with the suture that are beneficial for knot tying. The stationary jaw may also be integral to the housing, while the movable jaw possesses a feature that the user contacts in order to actuate the jaw. The jaw element may be utilized only for the purpose of grasping suture or it can serve multiple purposes, for example, to act as a suture cutter.

### BRIEF DESCRIPTION OF THE DRAWING FIGURES

FIG. 1A is a side elevation view of a surgical tool in accordance with a first embodiment with a suture grasping mechanism being shown in a first open position;

FIG. 1B is a side elevation view of the surgical tool with the suture grasping mechanism being shown in a second closed position, whereby a suture is grasped;

FIG. 1C is a side elevation of the surgical tool with the suture grasping mechanism being shown back in its first open position;

FIG. 2A is a side elevation view with partial transparency to show the components of the suture grasping mechanism of FIG. 1A in the first open position;

FIG. 2B is a is a side elevation view with partial transparency to show the components of the suture grasping mechanism in the second closed position;

FIG. 3A is a side elevation view of a surgical tool in accordance with a second embodiment with the suture grasping mechanism being shown in a first closed position;

FIG. 3B is a side elevation view of the surgical tool of FIG. 3A with the suture grasping mechanism being shown in a second open position;

FIG. 3C is a side elevation view of the surgical tool of FIG. 3A with the suture grasping mechanism in the first closed position so as to capture a suture therein;

FIG. 3D is a side elevation view of the surgical tool of FIG. 3A with the suture grasping mechanism in the second open position for releasing the suture;

FIG. 4A is a side elevation view with partial transparency to show the components of the suture grasping mechanism of FIG. 3A in the first closed position;

FIG. 4B is a is a side elevation view with partial transparency to show the components of the suture grasping mechanism in the second open position;

FIG. 5A is a side elevation view of a surgical tool in accordance with a third embodiment with a suture grasping mechanism being shown in a first open position;

FIG. 5B is a side elevation view of the surgical tool of FIG. 5A with the suture grasping mechanism being shown in a second closed position;

FIG. 6A is a side elevation view of a surgical tool in accordance with a fourth embodiment with a suture grasping mechanism being shown in a first open position;

FIG. 6B is a side elevation view of the surgical tool of FIG. 6A with the suture grasping mechanism being shown in a second closed position;

FIG. 7A is a side elevation view of a surgical tool with a suture grasping mechanism that includes an integral cutter shown in an open position;

FIG. 7B is a side elevation view, in partial transparency, of the surgical tool of FIG. 7A showing the cutter in the open position;

FIG. 7C is a side elevation view, in partial transparency, of the surgical tool of FIG. 7A showing the cutter in the closed position;

FIG. 8A is a side elevation view of a surgical tool in accordance with one embodiment showing a suture wrapped loosely around the suture grasping mechanism;

FIG. 8B is a partial side elevation view showing the suture grasping mechanism in an open position;

FIG. 8C is a partial side elevation view showing the suture grasped by the suture grasping mechanism and manipulated so as to form a knot;

FIG. 8D illustrates the knot formed in the suture;

FIGS. 9A-9E show various exemplary suture grasping mechanisms with serrations and various textured jaws;

FIGS. 10-10D depict exemplary grooved jaws that comprise an exemplary suture grasping mechanism for capturing and manipulating a suture into a desired form, such as a loop;

FIG. 11A is a side elevation view of a surgical tool in accordance with another embodiment and including a retractable/extendable suture grasping mechanism being shown in a retracted position;

FIG. 11B is a side elevation view of the surgical tool of FIG. 11A with the suture grasping mechanism shown in an extended position with a suture grasper in an open position;

FIG. 11C is a side elevation view of the surgical tool of FIG. 11A with the suture grasping mechanism shown in the extended position with the suture grasper in a closed position;

FIG. 12A is a side elevation view with partial transparency to show the components of the suture grasping mechanism of FIG. 11A in the retracted position;

FIG. 12B is a side elevation view with partial transparency to show the components of the suture grasping mechanism of FIG. 11B in the extended position with the suture grasper in the open position;

FIG. 12C is a side elevation view with partial transparency to show the components of the suture grasping mechanism of FIG. 11C in the extended position with the suture grasper in the closed position;

FIG. 13A is a partial left side view of one end of the surgical device showing the suture grasping mechanism in the retracted position;

FIG. 13B is a partial left side view of one end of the surgical device showing the suture grasping mechanism in the extended position; and

FIG. 14 is a side elevation view of a surgical tool in accordance with another embodiment of the present invention.

### DETAILED DESCRIPTION OF CERTAIN EMBODIMENTS

Disclosed herein are device concepts and methods for managing suture in and near tissue, skin, muscle, ligament, tendon and similar structures throughout the entire body. Current procedures typically consist of a user utilizing a needle and suture with hemostats, a needle driver, forceps, or an engineered suturing device and then piercing the patient's tissue. Often, these instruments are used to manage and tie suture in addition to passing the needle through tissue.

A device according to one exemplary embodiment is a handheld suturing device with a grasping mechanism that can be used to manipulate and tie knots in a suture. The device utilizes a pre-loaded needle and suture or a user-loaded needle and suture. Further, it features an actuator mechanism for affecting the needle and suture assembly (a “needle transfer mechanism” or “needle shuttle mechanism”), and a suture grasping mechanism.

Many designs for suturing devices are readily found in commercial and public use, in patents and applications, and in literature. Some possess knot sliding and suture tying mechanisms while others do not. The suture grasping mechanism described herein could be coupled to the housing and/or be integral to the housing of any suture device or device that is used in a procedure or department that includes suturing.

The suture grasping mechanism can include an elongate stationary jaw and an elongate movable jaw, which when actuated, contacts the stationary jaw and grips the suture between the jaw faces. The movable jaw will separate from the stationary jaw when the actuation force is removed or reversed, thereby releasing the suture. The elongate nature of the jaws permits the user to precisely grasp and manipulate suture and to create loops with the suture that are beneficial for knot tying. The stationary jaw may also be integral to the housing, while the movable jaw possesses a feature that the user contacts to actuate the jaw. The jaw element may be utilized only for the purpose of grasping and managing suture or it can serve multiple purposes, for example, to also act as a suture cutter.

In an alternative form, the elongate stationary and movable jaws may be coapted in their at-rest condition and an actuation force causes the jaws to separate and be available to receive the suture.

In yet another form, the elongate jaws may both be movable and biased to a separated condition or a coapted condition. The user can affect the jaws in either configuration in order to grasp, release, tie, or affect the suture.

Looking more closely at one exemplary embodiment illustrated in the figures, a suturing device 100, comprises a handle 110 consisting of a housing 111, a preloaded or user-loaded needle and suture assembly 200, comprising a needle 210 and suture 220, and a suture grasping mechanism 130.

The handle 110, which can represent a multitude of commercial suturing devices is comprised of one or more components such as the housing 111 and actuator 112, may be molded, cast or extruded from a variety of materials including but not limited to polymers or metals. Examples of polymers suitable for fabricating the handle are thermoplastic and thermosetting materials such as polystyrene, acrylic, polycarbonate, polyamide, polyester, polyetherimide, polysulfone, polylactic acid, polyvinylchloride, polyolefins, polyurethane, fluoropolymers, and copolymers and alloys thereof. These materials may be filled with glass or other useful reinforcing agents in order to enhance their mechanical properties. Suitable metals come from but are not limited to a group including titanium alloys and stainless steel. The selected materials must meet physical and mechanical performance requirements and be able to withstand sterilization methods employed within the medical device industry such as ethylene oxide or gamma irradiation. The handle design may be constructed to be linear and longitudinal, non-planar, angled, arcuate or a combination of these conformations.

The needle assembly 200 generally consists of the needle 210 and the suture 220 attached thereto. The needle 210 includes a distal pointed end 211 suitable for piercing and crossing tissue and a blunt proximal end 212 suitable for affixing a suture, and a body between the distal and proximal ends. Alternatively, the needle may feature a point at both the distal and proximal ends. The suturing needle 210 can be fabricated in a variety of configurations from straight to curved and be monolithic, channel-bodied or of a multi-part construction. The outer diameters of the needles can be round or non-round, tapered, or possesses features that assist in advancing and gripping the needle, i.e., flats, ribs, corners. Longitudinal ribs or recessions or other features found on the outer diameter of the needle may provide additional rigidity, gripability and enhance the needle's ability to effectively cross tissue. Needles are commonly made from stainless steel and related alloys but can be made from other metals, polymers and ceramic materials that are sufficiently rigid, capable of possessing and sustaining a functionally sharp distal point, and able to attach to suture. Traditionally, sutures are affixed to the proximal end of metal needles by swaging, crimping, knotting and adhesives. Suture attachment can also be configured such that the suture is affixed to the other regions of the needle, yet not the proximal terminus. This design variant provides additional freedom for suture management and gripping the needle in the device handle. In these configurations, attachment of the suture can be made by swaging, crimping, knotting, adhesives, etc. Coatings on the needle including but not limited to silicone, polyethylene glycol and/or glycerin serve to enhance the lubricity of the needle and reduce tissue penetration forces.

The suture 220 is the thread-like material that is used to treat internal and external wounds and incisions and to secure catheters or other components to patients. It comes in a variety of diameters, textures, forms, i.e., single strand or braided, and materials depending upon the desired properties and intended application. Sutures 220 can be absorbable, i.e., collagen, polyglactin, polydioxanone, polyglycolide-lactide copolymers, or non-absorbable, i.e., silk, nylon, polyester, polypropylene, stainless steel. They can be treated and/or coated with antimicrobial (e.g., chlorhexidine, silver, triclosan), bioabsorbable (e.g., glycolide/trimethylene carbonate, hydrogels, polyethylene oxide), hydrophilic (e.g., polycaprolactone, polyethyelene oxide), lubricious (e.g., silicone, polyethylene glycol, glycerin) or other functional additives. In addition, they can have surface features, e.g., barbs, that permit the suture to be drawn smoothly through tissue in one direction but snag the tissue when pulled in the opposite direction. This is advantageous when the user wants to temporarily or permanently approximate tissue without the need to tie a traditional knot.

It will be appreciated that the needle assembly 200 can be part of a suturing mechanism that can be operated to effectuate suturing of the tissue with the needle 210. It will be understood that any number of suturing mechanisms can be used as part of one of the suturing devices disclosed herein. For example, exemplary suturing mechanisms for the suturing device 100 are disclosed in commonly owned U.S. Pat. Nos. 9,125,644; 9,326,765; 9,554,793; 9,743,924; and US Patent application publication No. 2018/0153540, each of which is hereby expressly incorporated herein in its entirety.

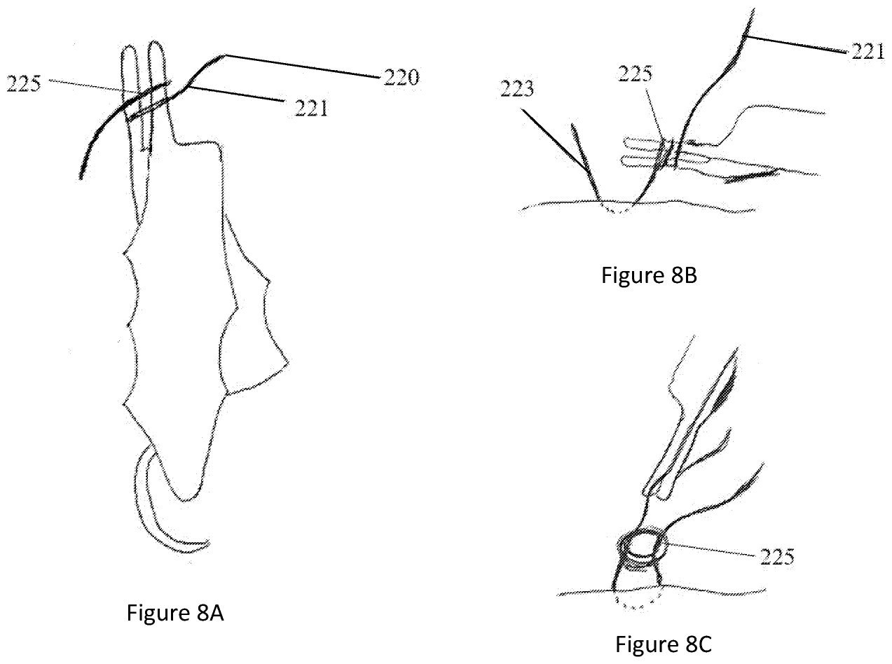

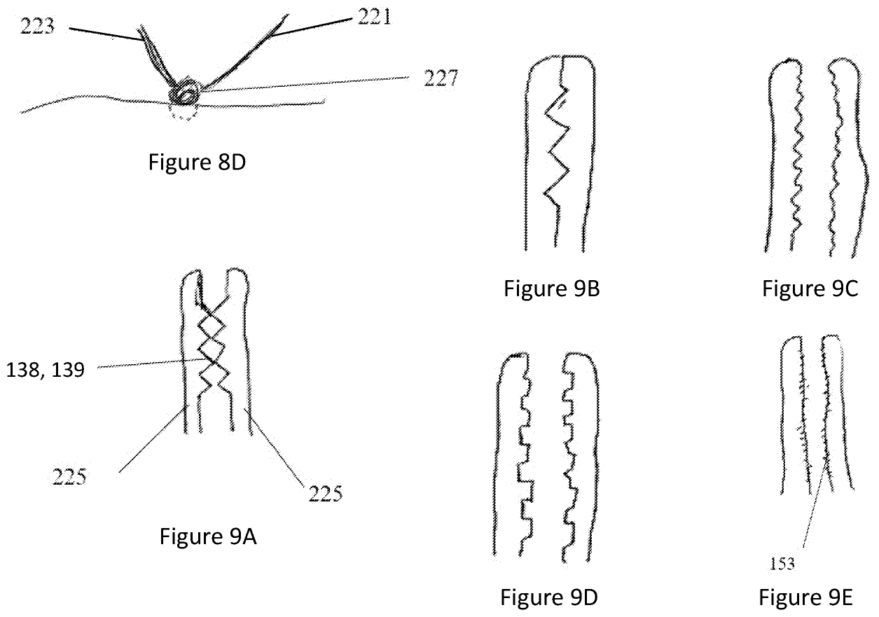

The suture grasping mechanism 130 can comprise an elongate stationary jaw 131 and an elongate movable jaw 132, which when actuated by the user, contacts the stationary jaw 131 and grips the suture 220 between the jaw faces 133, 134. The movable jaw 132 will separate from (move away from) the stationary jaw 131 when the actuation force is removed or reversed (i.e., when the user releases the jaw 132), thereby releasing the suture 122. The elongate nature of the jaws 131, 132 permits the user to create loops with the suture 220 that are beneficial for knot 227 tying (FIG. 8D). The stationary jaw 131 is coupled to the housing 111, while the movable jaw 132 possesses a feature 135, such as a button, pad, lever, etc., that the user contacts in order to actuate the jaw 132. This actuation may be configured to be rotational, linear or some other orientation suitable for actuation by the user. In one example, the movable jaw 132 is rotationally coupled to the housing 111 via a pivot 136 and biased to an open, suture-receiving and releasing condition by a spring 137 or similar means.

The elongate jaws 131, 132 are designed to affect the suture 220 in multiple ways, foremost to coapt and grip, open and release, and form wraps, twists or loops. These manipulations are facilitated by design features present on the jaws 131, 132. Tactile gripping of the suture 220 is enhanced by serrations 138, 139 (FIG. 9A) on the jaw faces 133, 134. These serrations 138, 139 may be interlocking or not. Grip strength is created by the user's applied actuation force and enhanced by the material stiffness of the jaws 131, 132. Grip strength may also be increased by pre-loading the jaws 131, 132 such that the jaw faces 133, 134 contact each other before the travel of the movable jaw 132 is completed. This premature contact point creates a greater force for suture 220 gripping as the generally rigid jaws 131, 132 are subjected to an increasing contact load. Those skilled in the art will also appreciate the numerous methods that can be used for creating leverage and mechanical advantage in order to increase the gripping strength of the jaws, e g , cams, lead screws, levers, gears.

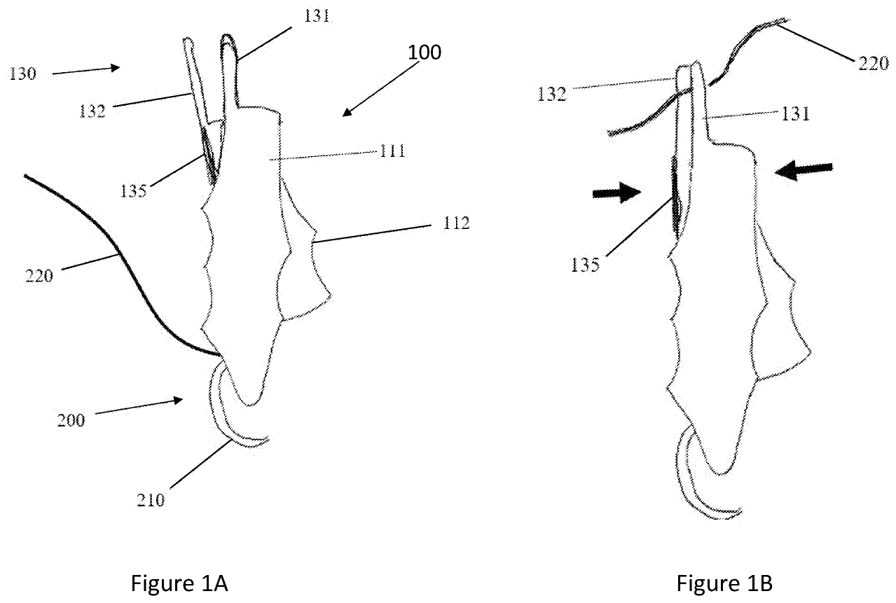

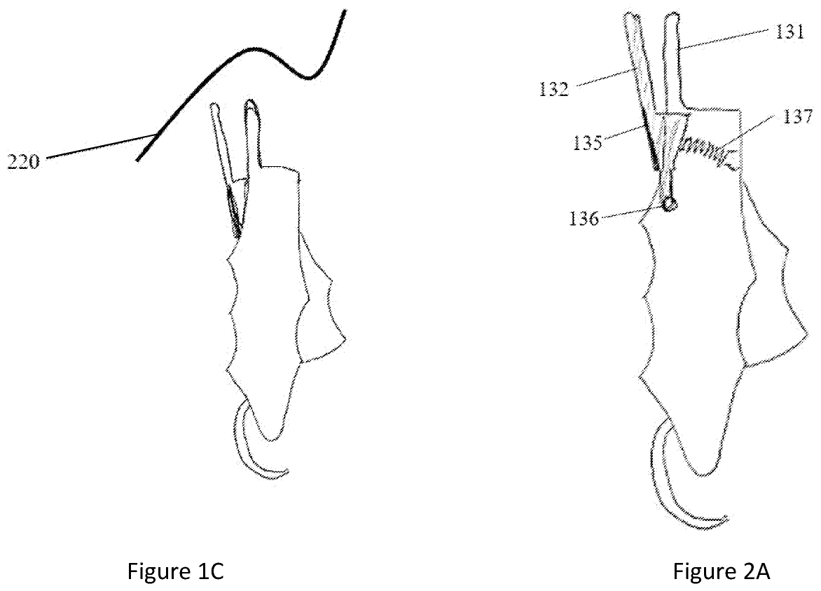

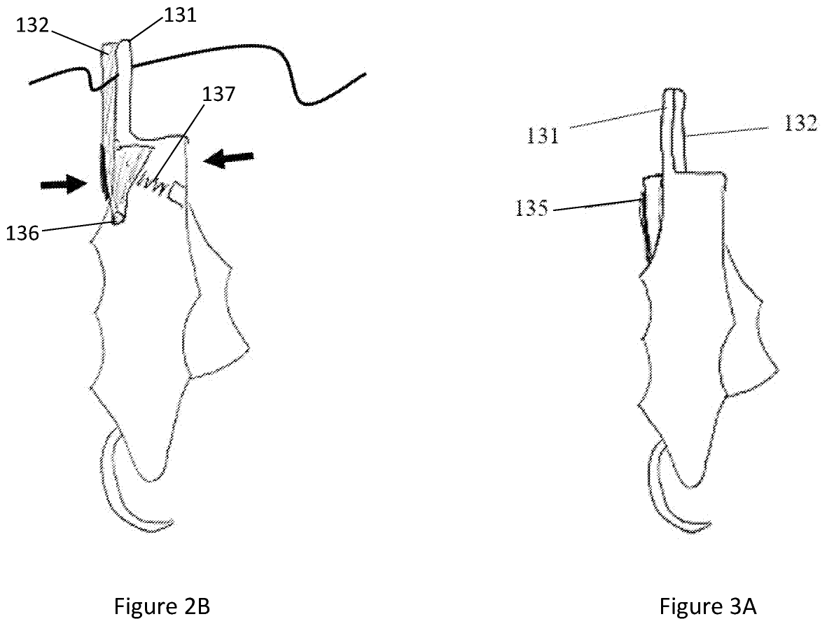

Referring to FIG. 1A, the suturing device 100 comprises the housing 111 and the suture grasping mechanism 130, which comprises the elongate stationary jaw 131 and the elongate movable jaw 132, shown biased to an open position, ready to affect suture 220. For the sake of clarity in subsequent figures, suture 220 is not shown attached to the needle 210. FIG. 1B depicts the suture 220 being grasped between the movable jaw 132 and stationary jaw 131 as a result of the user squeezing the two jaws 131, 132 together using feature 135 and the housing 111. This results in the movable jaw 132 rotating or sliding towards the stationary jaw 131. Serrations 138, 139 (shown in FIG. 9A) can be employed to enhance the gripping force exerted on the suture 220. In FIG. 1C, when the force is removed from the movable jaw 132, a spring 137 (shown in FIG. 2A) or other biasing means returns the movable jaw 132 to its origin away from the stationary jaw 131, to an open position, and releases the suture 220. The biasing force shown in FIG. 1C returns the suture grasping mechanism to the state shown in FIG. 1A.

It will be appreciated that the biasing element can be provided in any number of different structures, including but not limited to a structure that is formed integral to the housing, such as a leaf spring that is molded as a feature of the housing or the actuator (e.g., rotating lever, etc.).

Now looking at FIG. 2A, the internal elements of the grasping mechanism portrayed in FIGS. 1A through 1C are shown. The stationary jaw 131 is rigidly coupled to the housing 111 of the suturing device 100 and the movable jaw 132 is pivotally mounted to the housing 111 at a pivot location 136. A spring 137 biases the movable jaw 132 to a default, open position. FIG. 2B depicts the movable jaw 132 and stationary jaw 131 in a coapted condition as the user squeezes the jaws 131, 132 together via feature 135 and the housing 111, thereby capturing the suture 220. Releasing the squeezing force will release the suture as the jaws 131, 132 return to an open condition via the compressed spring 137 translating the movable jaw 132 away from the stationary jaw 131.

FIGS. 2A and 2B show exemplary attachment points for spring 137 and in particular, the spring 137 (biasing force) is attached at one end to the movable jaw 132 and at the opposite end to the housing 111 (i.e., a fixed point). When the user squeezes, the movable jaw 132, the spring 137 compresses and stores energy and once the user releases the movable jaw 132, the energy is released and the movable jaw 132 returns to its at-rest (open) position.

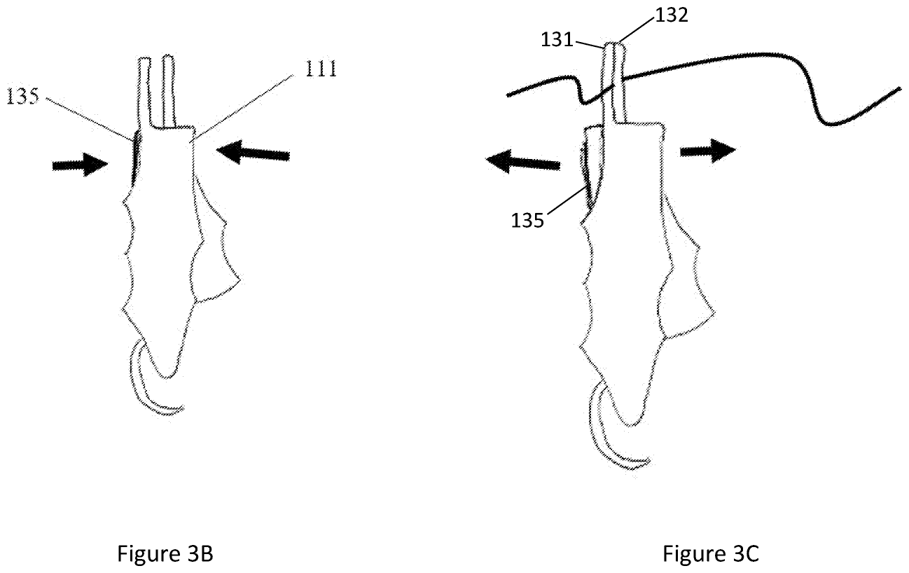

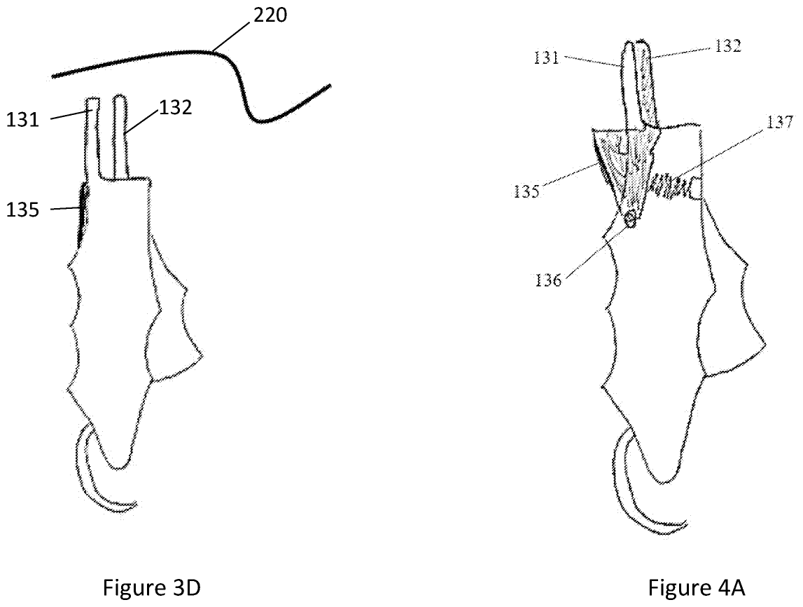

In an alternative form, the elongate stationary and movable jaws 132, 131 are coapted in their at-rest, default condition and an actuation force causes the jaws 131, 132 to separate and be available to receive the suture 220. FIGS. 3A through 3D illustrate this design variation. In FIG. 3A the jaws 131, 132 are at-rest and biased to this closed condition by spring 137 (FIG. 2A). As the user squeezes feature 135, and therefore the movable jaw, against housing 111, the movable jaw 132 travels away from stationary jaw 131 and creates an open jaw position capable of receiving suture 220 (see FIG. 3B). Next in FIG. 3C, the user removes the force from feature 135 and the spring 137 forces the jaws 131, 132 to the default, closed condition capable for grasping suture 220. Referring to FIG. 3D, depressing feature 135 again will open the jaws 131, 132 and release suture 220.

As described herein, the feature 135 can take the form of a structure that is contacted and manipulated in order to cause movement of the movable jaw. It will be appreciated that the embodiment of FIGS. 3A-3D depict an opposite arrangement of the jaws of the suture grasping mechanism in that in this embodiment, the jaws are biased to a closed position in the at-rest position in contrast to the first embodiment, in which the movable jaw is in an open position in the at-rest position.

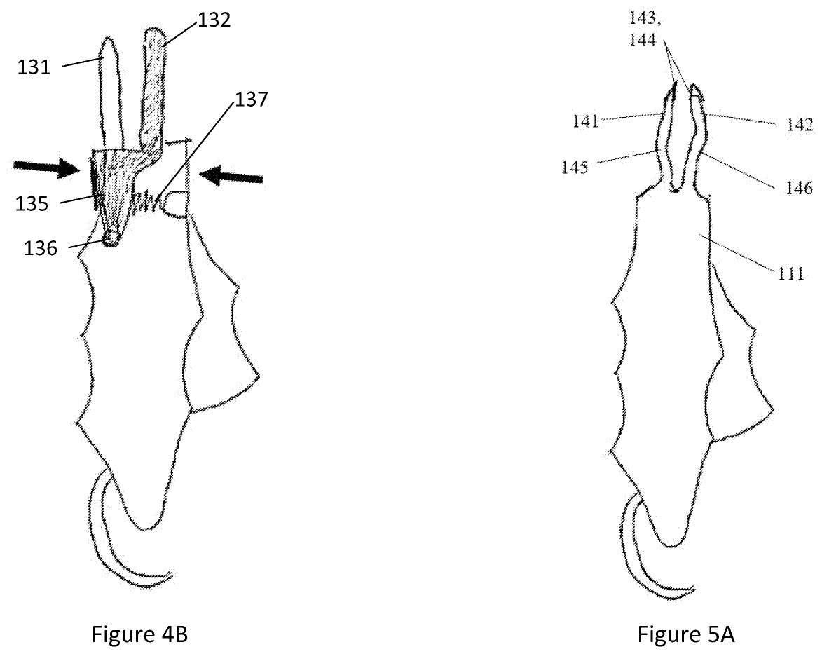

Now looking at FIG. 4A, the internal elements of the alternative grasping mechanism are shown. The stationary jaw 131 is rigidly coupled to the housing 111 of the suturing device 100 and the movable jaw 132 is pivotally mounted to the housing 111 at a pivot location 136. A spring 137 biases the movable jaw 132 to a default, closed position, which is suitable for grasping suture. FIG. 4B depicts the movable jaw 132 and stationary jaw 131 in an open condition, suitable for receiving or releasing suture, as the user squeezes the jaws 131, 132 together via feature 135 (e.g., a button or contact surface, etc.) and the housing 111. This sequence can be repeated by the user in order to purposely manipulate the suture.

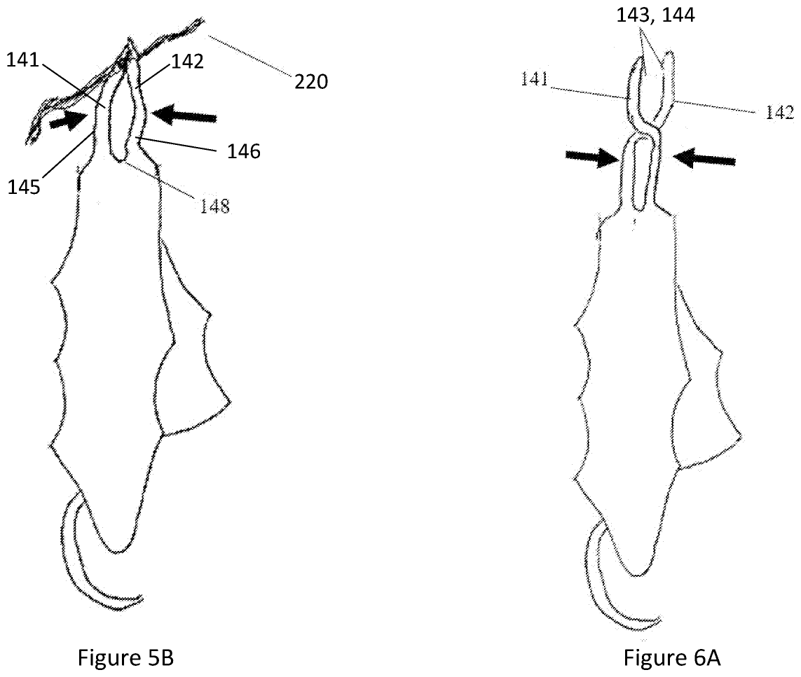

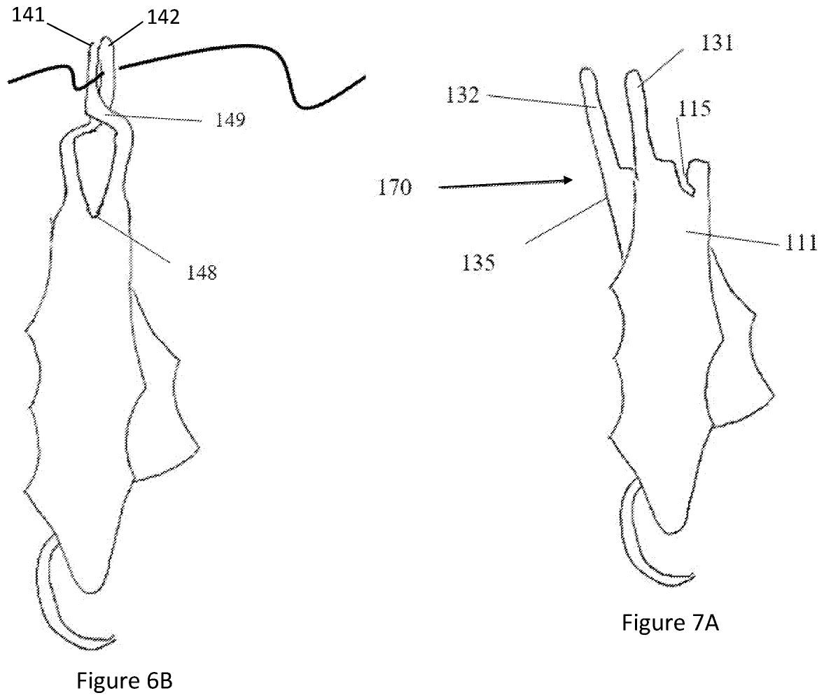

In yet another set of embodiments, the jaws 131, 132 as described above can both be configured to move, i.e., neither jaw is stationary, in order to grasp, release and affect suture 220. FIGS. 5A-5B and FIGS. 6A-6B depict a pair of movable semi-rigid jaws 141, 142 that can be opened or coapted through the action of squeezing or the removal of the squeezing force. These jaws can be constructed in a variety of ways to open and close symmetrically or unsymmetrically about the suture. p Looking specifically at FIG. 5A, one version of the jaws 141, 142 is depicted. These jaws are coupled to the housing 111 and default to a jaws-open condition. They are fabricated such that the user can easily manipulate them and they can firmly grasp the suture 220 between jaw faces 143, 144. In FIG. 5B, force applied simultaneously to actuation surfaces 145, 146 serve to coapt the jaws and grasp the suture. Removing this force permits the jaws 141, 142 to spring back to their default, open condition. This spring-like action can be achieved through the use of a biasing means, such as a spring or the elastic properties of the jaws' design and materials. For example, the jaws can be integral or coupled to the housing 111 and feature a flexible hinge point 148 capable of returning the jaws 141, 142 to their at-rest configuration. With respect to other variations of this and similar designs, one can easily envision a compression spring for example between the jaws, or also the individual jaws being pivotable about respective hinged joints. Typical materials of construction for the housing and/or jaws are polymers, e.g., polypropylene, nylon, acrylonitrile butadiene styrene, etc., and metals, e.g., spring steel, stainless steel, nickel-titanium alloys, etc. Another version for a pair of movable jaws 141, 142 is shown in FIGS. 6A and 6B, in which the jaws are designed to be in a default, coapted condition. FIG. 6A presents this specific embodiment at the moment when force is applied simultaneously to actuation surfaces 145, 146 which open the jaws to receive or release the suture 220. It shows the jaws' respective faces 143, 144, actuation surfaces 145, 146, and a pair of elastic hinge points 148, 149. Removing this force permits the jaws 141, 142 to spring back to their default, coapted condition as in FIG. 6B. Here the pair of jaws 141, 142, is in its coapted, default condition. This spring-like action can be achieved through the use of a biasing means, such as a spring or the elastic properties of the jaws' design and materials. For example, the jaws can be integral or coupled to the housing 111 and feature flexible hinge points 148, 149 capable of returning the jaws 141, 142 to their at-rest configuration. Typical materials of construction are polymers, e.g., polypropylene, nylon, acrylonitrile butadiene styrene, etc., and metals, e.g., spring steel, stainless steel, nickel-titanium alloys, etc.

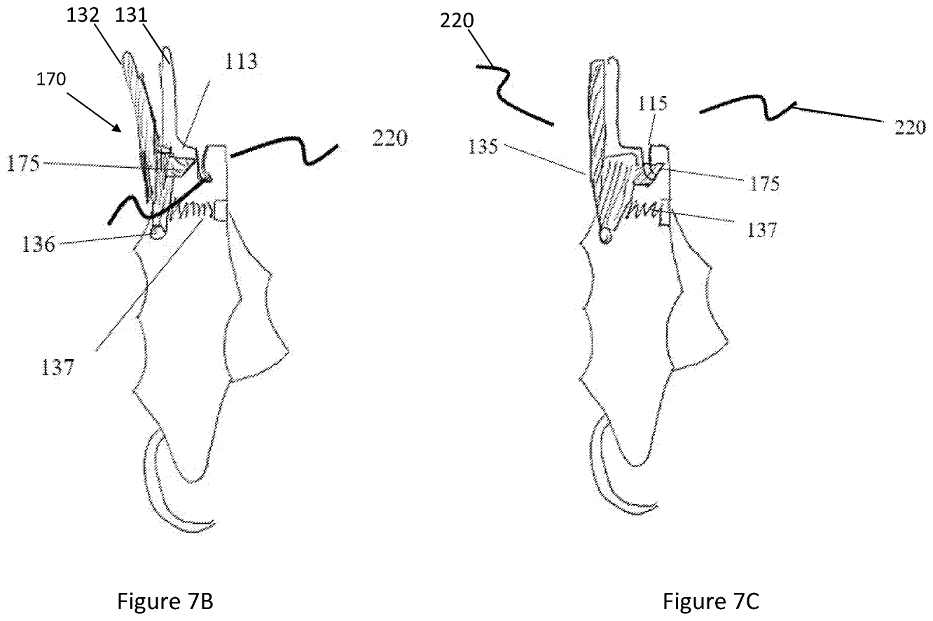

As mentioned above, the jaws 131, 132 can serve other purposes besides gripping, releasing, and knotting suture. A suture cutter 170 is referenced as an example of a concurrent mechanism. Consider FIGS. 7A through 7C in which a suture cutting mechanism 170 is integral to one of the suture grasping jaws 131, 132. As an example, the suture cutter body 171 will be detailed as an integral portion of the movable jaw 132; moving forward, they will be referenced as a composite. Referring to FIG. 7A, the suture cutter mechanism 170 comprises a cutter body 171 that is pivotally attached to the housing 111 at pivot 136 (not shown). The cutter body 171 holds a blade 175 (not shown) that faces inward toward the housing 111 and has the potential to traverse through a slot 113 in housing 111 and across a suture receiving notch 115, also formed by the housing. Note that the blade 175 can cut suture 220 that is positioned within suture receiving notch 115 when the blade traverses through slot 113 and across notch 115. A biasing member, such as a spring 137 (shown in a subsequent figure), is disposed within the housing 111 and biases the composite jaw 132 and suture cutter body 171 into a default, open condition in this example. It is in this condition that the user can, by means of squeezing the cutter body 171 and the housing 111, either cut suture when suture is placed into the notch 115 or grasp the suture in between the two jaws 131, 132. FIG. 7B depicts an internal view of the composite movable jaw 132 and suture cutter body 171 in the default open, suture receiving condition. The blade 175 is adjacent to the slot 113 and its distal portion is positioned on the edge of the notch 115. For clarity, slot 113 is parallel to the blade and serves as a guiding channel for the blade as it moves within the housing. As mentioned in previous paragraphs, the movable jaw 132 is displaced from the stationary jaw 131 when the device is at rest. Spring 137 and cutter body pivot 136 are also visible in this view. Furthermore, FIG. 7C demonstrates the configuration of the internal mechanisms when the user applies a force to the composite jaw 132 and suture cutter body 171 and it rotates about pivot 136. It should be noted that this embodiment describes a rotational travel of the jaw 132 and body 135, however, it is easy to envision the use of a linear track and actuation to accomplish the same end. The biasing spring 137 is compressed during the movement of the cutter body 171 as the blade 175 traverses along slot 113 and across suture receiving notch 115, and movable jaw 132 coapts with stationary jaw 131. Here the suture is shown cut into two segments. Removal of the user force allows the composite jaw 132 and suture cutter body 171 to return to its default, open condition.

Another use of the elongated jaws 131, 132, is presented in FIGS. 8A through 8D. One end 221 of suture 220 is shown in FIG. 8A being wrapped loosely around the coapted jaws 131, 132 by the user in order to form a loop(s) 225 suitable, for example, for suture knot 227 tying. FIG. 8B depicts jaws 131, 132 in an open condition, due to the user removing the squeezing force from the jaws and the biasing element 137 translating the movable jaw 132 away from the stationary jaw 131. The jaws 131, 132 are ready to grasp another end 223 of the suture 220. As shown in FIG. 8C, the user has squeezed the jaws 131, 132 together via feature 135 and the housing 111, grasped suture end 223, and pulled it through the loop(s) 225 in order to form a knot 227, which can be seen in FIG. 8D. This sequence of steps can be repeated in order, for example, to increase the security and strength of the specific knot or to create another separate knot.

Looking specifically at the jaw design, serrations 138, 139 and/or texture 153 can be incorporated onto one or both jaw faces 133, 134 in order to enhance suture gripping strength. Of course, the faces of the jaws could be flat and smooth. Serrations serve to lock or pinch the suture 220 through the use of physical peaks and valleys. Texture is used to increase the frictional nature of the jaw faces. A few examples of the many possible serrations and textures are shown in FIGS. 9A through 9E. Respectively, these figures present serrations that are non-interlocking, interlocking, radiused, square, and textured. These serrations can be formed through molding, stamping, knurling, or any other operation capable of creating these features. Texturing can be achieved through molding, dipping, spraying on coatings, e.g., rubber, elastomer, adhesive, etc., or through creation of a roughened surface by way of molding, embossing, machining, chemical etching, etc.

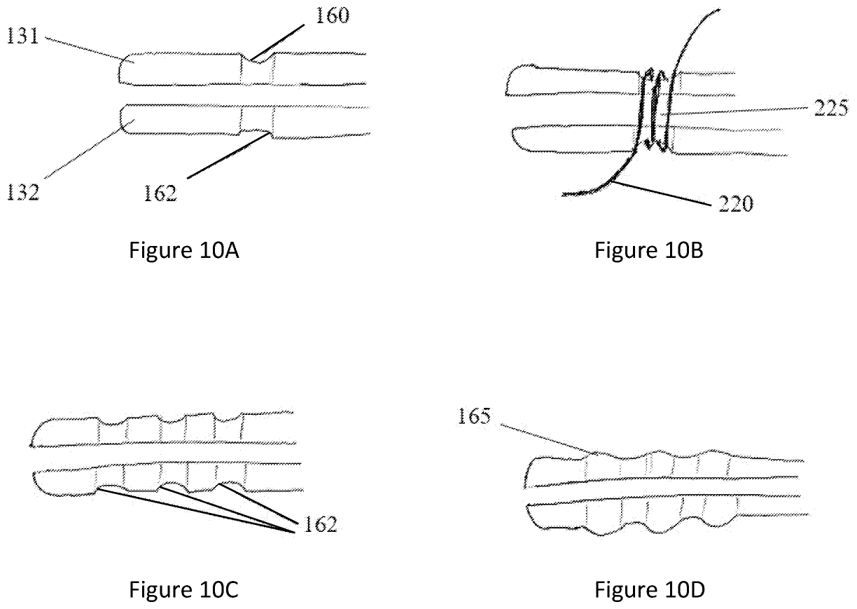

An additional aspect of the jaw design that can facilitate knot 227 tying is the incorporation of features such as a groove(s) 160, 162 located along the length of one or both jaws 131,132. The groove(s) 160, 162 serves to locate and control the position of the suture 220 during the loop 225 forming and knot tying processes. This provides the user with greater dexterity when handling the suture by minimizing the opportunity for the loop to accidentally slip off of the jaws 131, 132. In one embodiment the groove may be circumferential as initially presented in FIG. 10A. It can be shallow or deep, narrow or wide, radiused or cornered. Looking now at FIG. 10B, a suture end 221 has been wrapped around the jaws to form a loop 225 and is situated in the groove(s) 160, 162. One should be mindful that the features can be single or multiple grooves (FIG. 10C) or raised structures such as bumps or ribs 165 (FIG. 10D). These are only a few of the possible configurations and geometries for affecting the suture.

It should be understood that knot tying with this invention can be facilitated by either a jaws-open default or a jaws-closed default device, and with either one or more movable jaws. It should also be understood that the relative movement between each jaw 131, 132, 141, 142 and between the housing 111 can be rotational, linear or some combination of the two. Further, the design, length, appearance, and stiffness of the jaws can be constructed in numerous ways in order to better address a specific application.

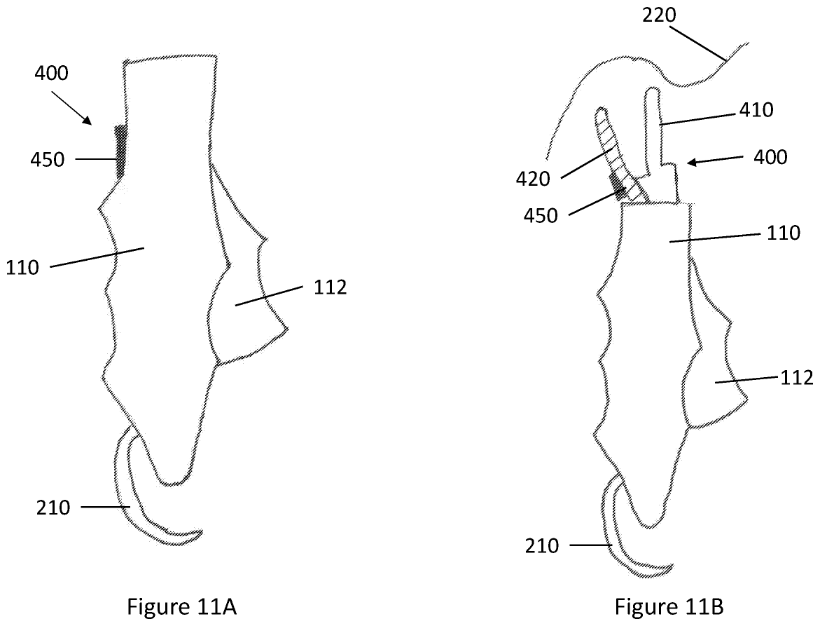

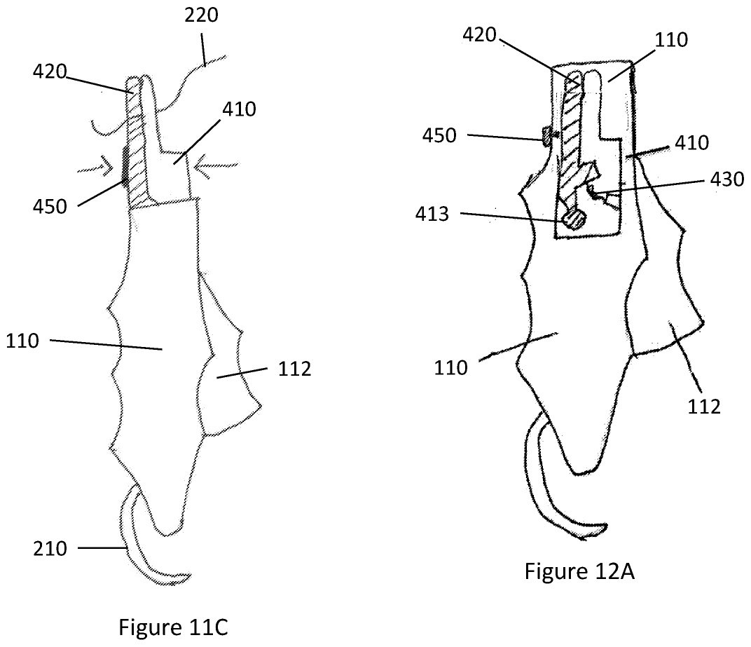

FIGS. 11A-13B illustrate one exemplary suture grasping mechanism 400 that can be incorporated into any of the surgical devices described herein including surgical device 100. In particular, FIGS. 11A-13B show suture grasping mechanism 400 incorporated into handle 110. Other elements of the surgical device shown in FIGS. 11A-13B that are in common with previous embodiments are numbered alike and in particular, actuator 112 and needle 210 are included.

The suture grasping mechanism 400 is of a retractable/extendable type relative to the housing 110. The suture grasping mechanism 400 can include a fixed (first) jaw 410 (shown in FIG. 11B) (similar to fixed (stationary) jaw 131) and a movable (second) jaw 420 (similar to movable jaw 132). More specifically, FIG. 11A shows the suture grasping mechanism 400 in a retracted position (storage condition of the device) with the movable jaw 420 being in a closed position. FIG. 11B shows the suture grasping mechanism 400 in an extended (extracted) position with the movable jaw 420 in an open position relative to the fixed jaw 410. FIG. 11B thus depicts the translation of both the movable jaw 420 and stationary jaw 410 to the extended position with the movable jaw 420, shown biased to an open position, ready to affect suture.

FIG. 11C shows the suture grasping mechanism 400 in the extended position with a force being applied (indicated by arrows) to the movable jaw 420 to effectuate closing of the movable jaw 420 and capturing of the suture element 220 between the two jaws 410, 420. This action is accomplished as a result of the user squeezing the two jaws 410, 420 using an actuator (e.g., lever) 450 and the stationary jaw 410. When the force is removed from the movable jaw 420, a spring (not shown in this figure) or other biasing means returns the movable jaw 420 to its open position.

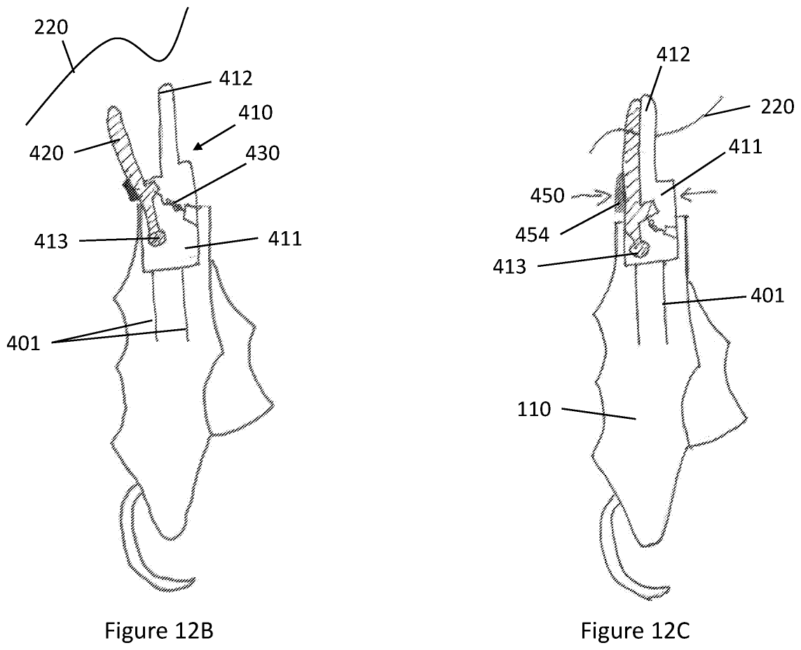

FIGS. 12A-12C show one exemplary method for extending and retracting the suture grasping mechanism 400 relative to the handle 110. More specifically, the housing of the handle 110 includes an opening formed therein and in which the suture grasping mechanism 400 can travel. In the illustrated embodiment, the handle 110 is open along its proximal end. Internally within the handle 110, there can be one or more guide rails 401 or the like to guide the movement of the suture grasping mechanism 400. The stationary jaw 410 can include a base portion 411 and a finger portion 412 that extends from the base portion 411. The base portion 411 can be the portion that rides along the guide rails 401. It will be appreciated that one or more stops can be incorporated into the design of the handle 110 to control and limit the movement of the suture grasping mechanism 400. For example, a first stop will limit downward movement within the handle housing, while a second stop will limit upward movement within the handle housing and prevent separation of the suture grasping mechanism 400 from the handle 110. Other mechanical features, such as detents and the like, can be included to properly ensure that the suture grasping mechanism 400 moves in a controlled manner within the interior of the handle.

FIGS. 12A-12C show linear movement of the suture grasping mechanism 400; however, other types of motion are envisioned. The movable jaw 420 is movably coupled to the stationary jaw 410 as by being pivotally coupled to the base portion 411 at a pivot 413.

The movable jaw 420 is also biased by a biasing element 430 which can be in the form of a spring, such as a compression spring or the like. The biasing element 430 is coupled at one end to the movable jaw 420 and at an opposite end to another structure, such as the base portion 411 or even the housing of the handle 110. In the illustrated embodiment, the biasing element 430 serves to bias the movable jaw 420 to an open position relative to the stationary jaw 410 as shown in FIG. 12B. However, as described previously, the biasing element can be configured to perform an opposite operation and bias the movable jaw to a closed position (closed at rest).

In FIG. 12A, the suture grasping mechanism 400 is in the fully retracted position and the movable jaw 420 is closed. The biasing element 430 is storing energy in this position.

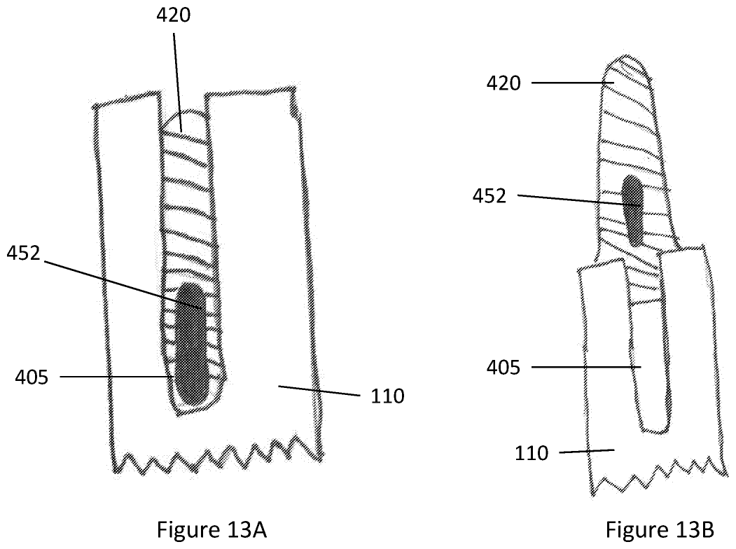

The user then moves the suture grasping mechanism 400 to the fully extended position by applying a driving force to the mechanism 400 as by using an actuator 450 (as described below with respect to FIGS. 13A and 13B) to cause the mechanism 400 to extend and protrude from the housing. As the mechanism 400 moves in this direction, the two jaws 410, 420 are incrementally revealed and the biasing element's stored energy is released to cause the movable jaw 420 to pivot to its open position as shown in FIG. 12B. The suture element 220 can be inserted between the two jaws 410, 420. FIG. 12B thus depicts the translation of the two jaws 410, 420 using the internal guide rails to guide the two jaws 410, 420 while the user applies a sliding force in the distal direction. The movable jaw 420 is shown biased open by use of the biasing element.

To close the mechanism 400, the movable jaw 420 is drawn towards the fixed jaw 410 as shown in FIG. 12C as by applying an inward force to the jaw 420 (see directional arrows) resulting in the movable jaw 420 moving toward and into contact with jaw 410 and the suture element 220 being captured therebetween. Energy is stored in the biasing element 430 in this position. FIG. 12C thus depicts the suture 220 being grasped between the movable and stationary jaws 420, 410 as a result of the user squeezing the two jaws 420, 410 and compressing the biasing element 430 using the lever feature 450. Releasing the squeeze force will release the suture as the jaws 420, 410 return to an open condition via the compressed biasing element 430 translating the movable jaw 420 away from the stationary jaw 410.

To retract the mechanism 400, a user simply applies a downward force to the unit and the mechanism 400 travels into the interior of the handle 110. It will be understood that mechanical features, such as detents and complementary structures, can serve to releasably secure the mechanism 400 in the fully retracted position and also optionally in the fully extended position. This linear movement of mechanism 400 is akin to the blade movement of a utility knife.

FIGS. 13A and 13B show a side elevation of the handle 110 at one end. At this end at which the mechanism 400 is located, a slot 405 is formed in the handle housing and can be open at one end and closed at the opposite end. Actuator 450 can include a stem portion 452 that is sized to pass through and be contained within the slot 405 and an enlarged head portion 454 is formed at an outer end of the stem portion 452 and comprises the portion of the actuator that is contacted by the user (in this sense the actuator 450 can have a T shape). The head portion 454 is designed to be contacted by a thumb or finger of the user to cause linear movement of the mechanism 400 as by moving the actuator 450 linearly within the slot 405. It will be appreciated that other types of actuators can equally be used. FIG. 13A reflects the storage condition in which the actuator 450 is in the proximal most position. In FIG. 13B, the mechanism 400 is extended and the movable jaw 420 is shown in the extended position with the actuator 450 being free of the slot 405. Thus, FIG. 13B depicts the two jaws 410, 420 having been translated to the retracted position and the actuator (lever) has been translated to the distal most position. The translation will occur when the user applies a sliding force to the lever (actuator 450) in the proximal direction. To place the two jaws 410, 420 in the storage condition the user will apply a sliding force in the distal direction until the lever contacts the stop created by the housing.

For simplicity sake, FIGS. 13A and 13B do not show the enlarged head portion 454 (see FIG. 12C).

In addition, it will also be appreciated that while FIGS. 11A to 13B show the first jaw 410 as being a fixed or stationary jaw, it can also be configured to move like movable jaw 420 similar to what is shown in previous embodiments. In addition, the default position of the first jaw 410 and second jaw 420 can vary. For example, various embodiments include, but are not limited to: (1) the first jaw 410 is fixed, while the second jaw 420 is a movable jaw defaulted to an open position; (2) the first jaw 410 is fixed, while the second jaw 420 is a movable jaw defaulted to a closed position; (3) first and second jaws 410, 420 are movable jaws defaulted to the open position; and (4) first and second jaws 410, 420 are movable jaws defaulted to the closed position.

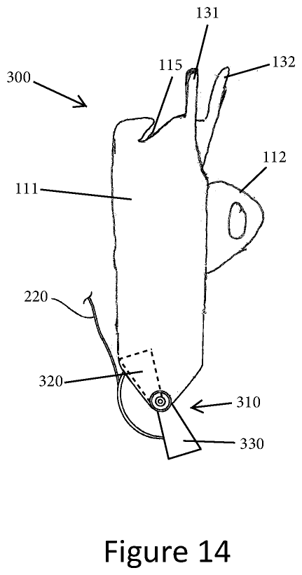

FIG. 14 depicts a suturing device 300 that is similar to the suturing device 100 and therefore, like elements are numbered alike. One difference between suturing device 300 and the suturing device 100 is that in the suturing device 300, the actuator 112 is formed along the same side of the housing 111 as the suture grasping mechanism 130. In addition, a suturing mechanism 310 (formed of one or more parts) of the suturing device 300 is as disclosed in commonly owned U.S. Pat. Nos. 9,125,644; 9,326,765; 9,554,793; 9,743,924; and US Patent application publication No. 2018/0153540, each of which has been previously incorporated by reference. The suturing mechanism 310 comprises the suturing needle 210, as well as, a first needle gripper 320 coupled to the handle 110 (housing 111) and a second needle gripper 330 coupled to the handle 110 (housing 111).

The first needle gripper 320 has an open position in which the suturing needle 210 can freely move relative thereto and a closed position in which the suturing needle 210 is held by the first needle gripper 320.

The second needle gripper 330 is movable relative to the handle 110 and has an open position in which the suturing needle 210 can freely move relative thereto and a closed position in which in the suturing needle 210 is held by the second needle gripper 330. The actuator 112 is operatively coupled to the second needle gripper 330 such that actuation of the actuator 112 rotates the second needle gripper 330 relative to the handle 110 and causes the first needle gripper 320 to assume one of the open and closed positions and causes the second needle gripper 330 to assume the other of the open and closed positions. Additional details and operation of the suturing mechanism 310 is found in the commonly owned US Patents and US published application previously identified herein.

FIG. 14 also shows a suture grasping mechanism such as any of the ones disclosed herein. For example, the illustrated suture grasping mechanism is the same or similar to the one described with respect to FIGS. 7A and 7B and includes slot 115, the fixed (stationary) jaw 131 and the movable jaw 132. As in FIG. 7B, the movable jaw 132 carries the blade that cuts the suture 220. It will be appreciated that any of the other suture grasping mechanisms, such as the one shown in FIG. 1A (that is not incorporated into a suture cutter mechanism) can equally be used. The suture grasping mechanism is shown at one end of the handle, while the suturing mechanism 310 is at the opposite end.

It will also be appreciated that additional features can be included as part of any of the suture grasping mechanisms disclosed herein. For example, the grasping jaws can be configured to lock and unlock. The locking capability allows the suture grasping mechanism to be temporarily disabled by placing it in a locked position. Any number of different types of lock mechanisms can be used. For example, a lock pin, ratchet, yoke, or the like can be used to lock a grasping jaw in place.

While the suture grasping mechanism has been described herein as being part of a device that also has a suturing mechanism that at least includes a suturing needle, it will be appreciated that the suture grasping mechanism can be part of a hand-held device (surgical tool or instrument) that does not include a suturing mechanism (e.g., the suturing mechanism can be part of another separate surgical device).

Although it is contemplated as a single-use device, it is understood that slight alterations can be made to the design and materials that would allow said device to be resterilized, reloaded with an additional needle and suture, or blade, and reused. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A surgical device comprising:
a handle having a housing, the handle being configured to perform at least a first operation; and
a suture grasping mechanism that forms part of the handle and includes a first part and a second part that is movable relative to the first part and the housing to perform a second operation different than the first operation and permit the suture grasping mechanism to have an open position for receiving a suture and a closed position for grasping and holding the suture between the first part and the second part.

2. A device for suturing tissue comprising:
a handle having a housing;
a suturing mechanism coupled to the handle and being configured to suture the tissue with a suture element; and
a suture grasping mechanism coupled to the housing and being configured to move between an open position for receiving the suture element and a closed position for grasping and holding the suture element.

3. The device of claim 2, wherein the suturing mechanism includes a suturing needle that can be driven through the tissue,

4. The device of claim 2, wherein the suturing mechanism is disposed at a first end of the handle and the suture grasping mechanism is disposed at or proximate to an opposite second end.

5. The device of claim 2, wherein the suturing mechanism includes an actuator that is disposed along a same side of the housing as the suture grasping mechanism.

6. The device of claim 2, wherein the suturing mechanism includes an actuator that is disposed along an opposite side of the housing as the suture grasping mechanism.

7. The device of claim 2, wherein the suture grasping mechanism includes a first jaw and a second jaw that moves relative to the first jaw, wherein in the open position, the first jaw and the second jaw are spaced apart from one another, while in the closed position, the first jaw abuts or is in close proximate contact with the second jaw.

8. The device of claim 7, wherein the first jaw comprises a movable jaw that is movably coupled to the housing, while the second jaw comprises a fixed jaw that is coupled to the housing.

9. The device of claim 7, wherein the first jaw is biased by a first biasing member that is coupled to the first jaw and the housing.

10. The device of claim 9, wherein the first biasing member comprises a spring that is attached at a first end to the first jaw and at a second end to the housing.

11. The device of claim 9, wherein the first jaw is biased to an open position which comprises an at-rest position of the suture grasping mechanism.

12. The device of claim 9, wherein the first jaw is biased to a closed position which comprise an at-rest position of the suture grasping mechanism.

13. The device of claim 12, wherein the suture grasping mechanism includes an actuator that when actuated causes the first jaw to move to the open position from the closed position whereby the first biasing mechanism stores energy.

14. The device of claim 2, wherein the suture grasping mechanism includes a first jaw and a second jaw that moves relative to the first jaw and further includes an actuator that comprises at least one of a button or lever that is disposed and accessible along the handle and configured to move the second jaw.

15. The device of claim 7, wherein each of the first jaw and the second jaw comprise a movable jaw.

16. The device of claim 15, wherein the first jaw and the second jaw flex about a hinge point.

17. The device of claim 15, wherein the first jaw and the second jaw assume the closed position in an at-rest position and include a pair of elastic hinge points about which the first jaw and the second jaw move.

18. The device of claim 2, further comprising a suture cutter formed including a slot formed within the housing for receiving the suture element.

19. The device of claim 18, wherein a part of the suture grasping mechanism also forms a part of the suture cutter and is configured to cut the suture element disposed within the slot.

20. The device of claim 19, wherein the part of the suture grasping mechanism that forms the part of the suture cutter comprises a movable jaw of the suture grasping mechanism that carries a blade, whereby movement of the movable jaw causes the suture cutter to move to a cutting position and be disposed at least partially within the slot and also concurrently causes the suture grasping mechanism to move between the open position and the closed position.

21. The device of claim 20, wherein the suture grasping mechanism further includes a fixed jaw that is part of the housing and in the closed position, the movable jaw moves in a direction toward the fixed jaw and the movable jaw carries a blade that enters an opening of the suture cutter configured to receive the suture element.

22. The device of claim 21, wherein the movable jaw is biased by a biasing member that extends between the movable jaw and at least one of the housing and the fixed jaw.

23. The device of claim 7, wherein at least one of the first jaw and the second jaw has a groove formed therein for receiving the suture element.

24. The device of claim 7, wherein at least one of the first jaw and the second jaw has a serrated surface for contacting the suture element.

25. The device of claim 2, wherein the suturing mechanism comprises:
a suturing needle having a first pointed end and an opposite second end;
a first needle gripper coupled to the handle, the first needle gripper having an open position in which the suturing needle can freely move relative thereto and a closed position in which the suturing needle is held by the first needle gripper;
a second needle gripper coupled to the handle, the second needle gripper being movable relative to the handle and having an open position in which the suturing needle can freely move relative thereto and a closed position in which in the suturing needle is held by the second needle gripper; and
an actuator that is operatively coupled to the second needle gripper, wherein actuation of the actuator rotates the second needle gripper relative to the handle, and causes the first needle gripper to assume one of the open and closed positions and causes the second needle gripper to assume the other of the open and closed positions.

26. The device of claim 25, wherein the actuator is a pivotable member located along one side of the housing and the suturing mechanism is disposed at a distal end of the housing and the suture grasping mechanism is disposed at a proximal end of the housing.

27. The device of claim 2, wherein the suture grasping mechanism moves between a retracted position and an extended position relative to the housing.

28. The device of claim 27, wherein the suture grasping mechanism includes a first jaw and a second jaw that moves relative to the first jaw, wherein in the open position, the first jaw and the second jaw are spaced apart from one another, while in the closed position, the first jaw abuts or is in close proximate contact with the second jaw, wherein in the retracted position, both the first jaw and the second jaw are at least substantially contained within a hollow interior of the housing.

29. The device of claim 28, wherein the first jaw includes a base portion that rides along one or more guide rails disposed within the housing and a protruding portion, the second jaw being pivotally mounted to the base portion of the first jaw.
